(19) United States
(12) Patent Application Publication
(10) Pub. No.: US 2016/0047805 A1
(43) Pub. Date: Feb. 18, 2016

(54) IN SITU-DILUTION METHOD AND SYSTEM FOR MEASURING MOLECULAR AND CHEMICAL INTERACTIONS

(71) Applicant: FLIR SYSTEMS, INC., Wilsonville, OR (US)

(72) Inventor: John Gerard Quinn, Edmond, OK (US)

(21) Appl. No.: 14/923,634

(22) Filed: Oct. 27, 2015

Related U.S. Application Data
(63) Continuation of application No. 13/521,788, filed on Feb. 12, 2013, now abandoned.
(60) Provisional application No. 61/294,747, filed on Jan. 13, 2010.

(51) Int. Cl. G01N 33/557

(52) U.S. Cl. CPC G01N 33/557

(57) ABSTRACT

The present invention relates to a method for testing multiple analyte concentrations within a biosensor system through a single injection of sample. The method involves flowing a fluid sample containing a neat analyte concentration along a flow path in a fluid system and diluting the sample by causing it to merge with a fluid that is free of analyte in a second flow path under laminar flow conditions. The merged fluid stream is directed through a turbulent third flow path of a very low dead volume. The third flow path carries the merged fluid stream to a sensing region where the analyte is exposed to an immobilized ligand. The concentration of analyte can be controlled in this method by adjusting the flow rates of the sample flow and analyte-free fluid flow. A fluidic system for carrying out this method is also disclosed.

### CROSS REFERENCE TO RELATED APPLICATIONS

The present application is a national phase entry of PCT Application No. PCT/US11/021190 entitled IN SITU-DILUTION METHOD AND SYSTEM FOR MEASURING MOLECULAR AND CHEMICAL INTERACTIONS filed Jan. 13, 2011, which claims priority to U.S. Provisional Patent Application Ser. No. 61/294,747 filed on Jan. 13, 2010.

### BACKGROUND OF INVENTION

Surface plasmon resonance (SPR) based biosensors are commonly used to perform kinetic studies of complex molecular interactions such as between hormone-receptor, enzyme-substrate and antigen-antibody. The biosensors are typically in the form of one or more sensing surfaces housed within a sensing region of a microfluidic system. The microfluidic system further defines a series of flow paths that direct fluid flow to the sensing region. The one or more sensing surfaces of the sensing region support immobilized molecules referred to as “ligands.” The ligands bind molecules known as “analytes” which are present in fluids that are directed to the sensing region via the microfluidic system. Current analysis methods determine the kinetic interaction of the ligand and analyte by separately injecting a series of analyte concentrations into the system and measuring the change in refractive index at the sensing region. Based on the changes in refractive index, one can determine the kinetics of the interaction between the analyte and ligand, including association and dissociation rates.

By assaying a wide range of analyte concentrations, one can establish a more accurate representation of the interaction kinetics. Traditionally, a range of analyte concentrations has been provided by the following methods: (1) separately injecting a series of test samples, each of increasing or decreasing analyte concentrations, into the microfluidic system (the “stepped injection method”); (2) injecting a single volume consisting of a concentration gradient (the “gradient injection method”); or (3) pulsing alternating segments of analyte containing sample and analyte-free sample at various rates which mix in route to the sensing region to yield a series of analyte concentrations (pulse method). Regardless of the analyte injection method, obtaining an accurate kinetic profile of the interaction requires an accurate representation of analyte concentrations at all time points during the assay.

The stepped injection method involves the preparation of an analyte dilution set, either manually, or using an on-board autosampler. In either case a series of additional sample positions are required. After dilution preparation, the samples are then loaded and injected separately where each analyte injection is either followed by a buffer injection or by a regeneration solution, depending on the method utilized. This is time consuming and provides ample opportunity for human error.

Furthermore, in the stepped injection method, the analyte concentration is not accurately represented during the transition zones when the analyte concentration is changing. In practice the concentration of analyte within the sensing region does not instantaneously become the injected analyte concentration once an injection commences. In reality the sample must “wash out” the dead volume and the sensing region by a factor of 2-3 equivalent volumes before the assumption of a steady-state fixed concentration is appropriate. Therefore these stepped injections should be considered stepped pseudo-fixed analyte injections where fixed analyte concentrations exist only after a steady state has developed. Thus, it would be preferable to fit an interaction model where the concentration of analyte within the sensing region is accurately represented at all times thereby allowing the interaction model to be fitted more accurately to the entire stepped analyte concentration curve.

The gradient injection method provides the benefit of avoiding multiple injections in that only a single injection is required to assay a wide range of analyte concentrations. However, this method requires the use of an external gradient maker. Furthermore, in a gradient injection, the analyte concentration is constantly changing. Thus, a steady state analyte concentration in the sensing region cannot be obtained.

The pulse method provides the benefit of avoiding the preparation of a dilution series or reliance on an external gradient maker. However, the pulse method relies heavily on dispersive mixing to generate the analyte concentrations. Accurate estimation of the analyte concentration at any point after significant dispersion takes place is a complex function of molecular weight, viscosity, ionic strength, isoelectric point, channel geometry, flow rate and cannot be described by Taylor Theory under the conditions considered optimum for biosensing applications. Therefore eliminating any dependence on “Taylor-like” dispersion in the flow channels is an effective approach to avoiding these difficult problems yet all the gradient injection methods described in the previous prior art rely heavily on this means of mixing. Thus, the analyte injection profiles are a significant function of molecular weight or other physical parameters of the analyte or fluidics. In some cases the inventors assume approximate relationships that can be applied under particular conditions that apply to special case applications and more general application is not possible.

In sum, these prior art methods are either time consuming or fail to provide an accurate representation of the analyte concentration in the sensing regions. Thus, a method is needed that decreases running time while providing an accurate representation of the analyte concentration in the sensing region at all times.

### SUMMARY

The current invention provides a biosensor-based method to perform kinetic analysis without preparation and injection of multiple samples or an external gradient maker. Furthermore, the current method does not rely on dispersion to yield a series of analyte concentrations.

Therefore, in one embodiment, the invention provides a method of exposing a sensing region in a biosensor system to a series of analyte concentration in order to perform kinetic analysis of molecular interactions comprising the steps of: passing a first fluid through a first flow path at a first fluid flow rate, said first fluid including a first concentration of analyte; passing a second fluid through a second flow path at a second fluid flow rate, said second fluid free of the analyte; merging the first and second fluids from said first and second flow paths into a third flow path to form a third fluid, said third fluid having a second concentration of analyte; passing said third fluid through said third flow path at a third fluid flow rate to a sensing region; and continuing the flow of said first, second and third fluids without interruption while adjusting the concentration of said analyte in said third fluid by adjusting the first and second fluid flow rates at least once thereby exposing the sensing region to at least one additional concentration of analyte in said third fluid different from said second concentration of analyte

In one aspect, the third flow path supports a low dead volume in order to minimize dispersion. In this aspect, the third flow path supports a volume of less than 5 μl.

In another aspect, the combined stream is mixed in the third flow path through chaotic advection and/or turbulence. In this aspect, the third flow path passes through one or more router valves prior to entering the sensing region.

In yet another aspect, the combined stream is maintained at a constant volumetric flow rate.

In another embodiment, the method further comprises the step of ceasing flow of the first fluid following exposure of the sensing region to all desired concentrations of analyte and allowing the second fluid to continue flow through the sensing region in order to measure dissociation of the analyte.

In another embodiment, the method further comprises the steps of: applying a fourth fluid to a fourth flow path, said fourth fluid free of analyte, wherein the fourth flow paths merges with the third flow path thereby resulting in further dilution of the third fluid.

In another embodiment, a fluidic system used to perform the in situ method is provided. The system comprises: at least two fluid actuating devices that are able to move a fluid along a flow path at a variety of flow rates, at least one output line in communication with each fluid actuation device; a junction where the output lines merge into a primary injection line. The primary injection line being in fluid communication with a sensing region where the sensing region has at least one sensing surface conducive for supporting an immobilized molecular or chemical entity.

In one aspect of the system, the primary injection line is of a low dead volume to prevent dispersion of the sample. In another aspect, the primary injection line has a volume capacity of 10 μL. Preferably, the primary injection line has a volume capacity of less than 5 μL.

In another aspect, the primary injection line provides a tortuous flow path to permit sufficient mixture of any fluids therein. In one aspect, the tortuous flow path includes running the primary injection line through a series of router valves and a microfluidic adapter.

Additional advantages and features of the current inventive method will become apparent from the following detailed description and drawings.

### BRIEF DESCRIPTION OF THE DRAWINGS

FIG. 1 is a schematic illustration of a system for performing the method of the current invention.

FIG. 2 is a schematic illustration of a fluidic system suitable for practicing the current invention with the sample router valve in the sample load position.

FIG. 3 is a schematic illustration of a fluidic system suitable for practicing the current invention with the sample router valve in the sample inject position.

FIG. 4A is a sensogram obtained by a stepped injection method of carboxybenenze sulfonamide over a sensing surface with immobilized carbonic anhydrase at 10° C.

FIG. 4B is a sensogram obtained by a stepped injection method of carboxybenenze sulfonamide over a sensing surface with immobilized carbonic anhydrase at 20° C.

FIG. 4C is a sensogram obtained by a stepped injection method of carboxybenenze sulfonamide over a sensing surface with immobilized carbonic anhydrase at 30° C.

FIG. 4D is a sensogram obtained by a stepped injection method of carboxybenenze sulfonamide over a sensing surface with immobilized carbonic anhydrase at 40° C.

FIG. 5A is a sensogram obtained by in situ dilution injection of carboxybenenze sulfonamide over a sensing surface with immobilized carbonic anhydrase at 10° C.

FIG. 5B is a sensogram obtained by in situ dilution injection of carboxybenenze sulfonamide over a sensing surface with immobilized carbonic anhydrase at 25° C.

FIG. 5C is a sensogram obtained by in situ dilution injection of carboxybenenze sulfonamide over a sensing surface with immobilized carbonic anhydrase at 35° C.

FIG. 6 is a sensogram demonstrating the bulk refractive index obtained by in situ dilution injection of DMSO.

FIG. 7 is a sensogram demonstrating the bulk refractive index obtained by in situ dilution injections of protein macroglobulin (molecular weight 725 kDa) and bovine serum albumin.

FIG. 8 is a sensogram demonstrating the bulk refractive index obtain by in situ dilution injection of DMSO.

FIG. 9 is a sensogram demonstrating the bulk refractive index obtain by in situ dilution stepped injection of DMSO.

### DETAILED DESCRIPTION OF THE PREFERRED EMBODIMENTS

The method and system described herein uses a single sample of analyte at a fixed concentration (“sample”) to establish interaction kinetics for a range of analyte concentrations. In the practice of the method, the user injects the sample into the fluidic system and controls the relative flow rates of sample and buffer streams to increase or decrease analyte concentration reaching the sensor region. As such, the method dispenses with separately preparing and injecting multiple sample dilutions, which saves a considerable amount of preparation time, space in sample plates and significantly decreases the probability of human error. For example, in an assay requiring an increasing analyte concentration, the flow rate of the sample (analyte-containing) stream is increased proportionately to a corresponding decrease in the buffer stream. This proportionate increase and decrease of flow rate provides a constant volume in the flow cell at all times. In summary, once analyte flow commences through the sensing region, it is not interrupted until all desired analyte concentrations have been tested. This significantly decreases the amount of time expended to assay a wide range of analyte concentrations.

As used herein, the term “analyte” can refer to any biomolecule (i.e. antigen, antibody, protein, nucleic acid, lipid, sugar) or any chemical that can be measured in the systems described herein and can be present in a fluid sample. Similarly, the term “ligand” can refer to any biomolecule (i.e. antigen, antibody, protein, nucleic acid, lipid, sugar) or any chemical that can be measured in the systems described herein and can be either immobilized on a surface or present in a fluid sample.

While the current invention is described in the context of a SPR-based system, it should be understood the inventive method could be employed in a variety of other detection systems, including any affinity based detection system where an analyte binds to a ligand immobilized on a sensing surface. Provided, however, that the interaction between ligand and analyte can be represented by measuring a change at the sensing surface.

In one embodiment, a fluidic system is provided that permits a series of analyte concentrations to be tested in a biosensor system through preparation of a single sample of analyte at a fixed concentration. In one embodiment of the inventive system, the entire sample is injected into a holding line within the fluidic system. A first pump causes the sample to move along a flow path, wherein the pump permits step-wise increases and decrease in flow rates. Concurrently, a second pump moves an analyte-free buffer along a second flow path and the flow rate of the analyte-free buffer can be controlled in the same manner as the sample. The first and second flow paths merge at a junction in the system and are directed into a third flow path. The third flow path is preferably of a very low dead volume. The sample and buffer are mixed in the third flow path to provide a merged stream. The merged stream moves along the flow path and enters a sensing region which contains a ligand immobilized on a sensing surface. The merged stream thus exposes the immobilized ligand to a first concentration of analyte. The pump flow rates can then be proportionately adjusted to alter the analyte concentration in the combined stream. By proportionately adjusting the flow rates, the merged stream volume in the third flow path and sensing region remains constant, which is of benefit under most assay conditions.

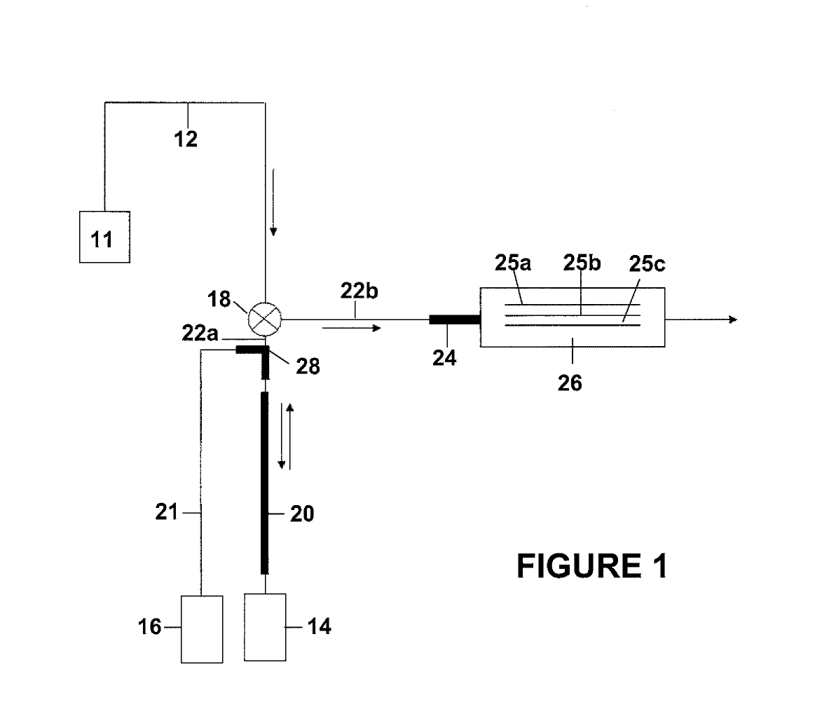

FIG. 1 represents a schematic by which to describe one embodiment of the current invention. Sample vial 11 containing a neat concentration of analyte (sample) is loaded into loading line 12 when the sample pump 14 aspirates with fluid router valve 18 in the load position. The sample is drawn past the router valve 18 into the holding line 20. Router valve 18 is then switched to the inject position and sample pump 14 dispenses the sample through injection line 22a through router valve 18 to injection line 22b and into the adapter 24 and then into the sensing region 26 containing sensing surfaces 25a, 25b, 25c having a ligand immobilized thereon (not depicted). Simultaneous with the above described injection of sample, buffer pump 16 will dispense buffer into line 21 causing both sample and buffer laminar flow streams in lines 20 and 21, respectively, to merge at the junction 28 and proceed into injection line 22a before entering router valve 18. The merged stream of buffer and sample progresses through router valve 18 where the buffer and sample mix as a result of the tortuous fluidic conduits (not depicted) present in the router valve 18 and adaptor 24 before entering the sensing region 26. Thus, one or more of the sensing surfaces 25a, 25b, and 25c are exposed to a diluted analyte concentration.

In one aspect, sample pump 14 may be off at the beginning of the method in order to obtain a zero analyte segment where analyte free buffer passes over sensing surfaces 25a, 25b, and 25c housed within sensing region 26. The flow rate of sample pump 14 and buffer pump 16 is set to the desired ratio required to provide the desired analyte concentration. For example, if 50% of the neat analyte concentration is desired, then the flow rates of pumps 14, 16 are matched. The laminar streams will be mixed in the low dead volume of injection line segments 22a and 22b before entering the sensing region 26.

In an alternative embodiment, it is possible to install an in-line microfluidic mixer (not depicted) before the sensing region 26. The microfluidic mixer could be placed between junction 28 and the router 18, but this is unnecessary if sufficient geometrical complexity exists in injection line segments 22a and 22b, router 18, and adaptor 24 to promote mixing of the merged stream in advance of arriving at the sensing region 26. Typically such router valves 18 and adapters 24 possess complex microfluidic paths that cause sufficient mixing of the merged laminar flow streams and thus, the microfluidic mixer will only be necessary if a system is employed to practice the current method that doesn't require router 18 and adapter 24.

In an alternative embodiment, it is possible to perform a primary and a secondary dilution of the neat analyte sample in advance of arriving at the sensing region 26 using the inventive method. This enables the analyte dilution range to exceed the flow resolution of the pump. In this embodiment, sample is loaded into loading line 12 when sample pump 14 aspirates with the fluid router valve 18 in the load position. Concurrently, buffer pump 16 delivers buffer into line 21 and both streams intersect at the junction 28 before the sample is completely loaded into holding line 20. Thus, the neat analyte concentration in the sample is initially diluted in route to holding line 20. The router valve 18 is then switched to the inject position and the sample pump 14 dispenses the diluted sample. When the sample pump 14 begins dispensing then the buffer pump 16 continues to dispense buffer such that the sample and buffer merge again at junction 28 and proceed through the system as described above.

In practicing this alternative embodiment, during the initial dilution as the sample is being loaded, it may be necessary for sample pump 14 to aspirate at a rate that is greater than the flow rate of buffer pump 16 in order to ensure that the streams merge smoothly thereby avoiding insertion of pulses (discrete segments) of buffer. Air bubbles can be employed to prevent dispersion of the sample during these manipulations. Accordingly, it is important to avoid turbulence at the junction that might cause bubble break up.

The flow rates that can be employed during sample aspiration usually greatly exceed the limits employed during sample injections. This results from restricting the flow rates through the sensing region path (injection line segments 22a, 22b, router 18, adapter 24) in order to avoid excessive backpressure. However, the microfluidic channels during sample loading, such as line 12 are far larger and can handle higher flow rates without excessive backpressure. Therefore a higher dilution range is possible during the sample loading phase. Thus, a combination of both dilutions (dual pass) can be applied where the sample passes the merging junction in both flow directions in order to accomplish a higher dilution range. A second holding line (not depicted) upstream from the merging junction 28 could be added to facilitate this method. In fact multiple passes are possible in order to accomplish very high dilution ranges. However a dilution range that covers 4-8 serial tripling dilutions is usually sufficient for kinetic analysis.

In another related embodiment, it is also possible to include more than one merging junction so that multiple analyte free streams merge with the sample stream enabling a higher dilution to be achieved. These strategies are favorable because any pump that might be chosen will have a lower flow rate limit and it is the flow rate range of the pump that determines the dilution range that can be accomplished in the in situ dilution method with acceptable error in analyte concentration.

For example, a syringe pump delivers a steady flow rate, but the flow rate will undulate very slightly (e.g. 0.1% of maximum flow rate). This undulation becomes proportionately greater at the lower limit of flow. Hence, the error when combining a slow flowing analyte stream with a fast analyte free buffer stream is more pronounced than when the flow rates are reversed. In one aspect of this embodiment, it is possible to overcome the flow limitation of the pump by incorporating a throttle mechanism where the actual flow at the merging junction 28 is less than the minimum pump flow rate in order to allow a higher dilution range to be accomplished. The throttle mechanism provides a means of accurately splitting the flow stream so that the pump can be operated well away from its resolution limit yet a very slow, and stable, flow rate can be delivered to the merging junction thereby extending the dilution range achievable.

For example, a basic throttle mechanism can be incorporated by simply adjusting the split flow ratio at a Y-junction by adjusting the relative channel geometry of each channel at the Y-junction or incorporation of a mechanically adjustable constriction. A mechanically variable orifice that can be actuated in response to the feedback signal from an in-line flow meter could also be used in combination with a Y-junction. The flow meter and orifice feedback control method would allow the flow rate through one arm of the Y-junction be controlled accurately and since the parent flow is usually well known all flow are known precisely.

In addition to merging a sample stream with a buffer (sample free liquid) stream it is sometimes desirable to perform merged dual sample injections where two separate sample streams containing different samples merge, and mix, before entering the sensing region. For example, competitive solution phase kinetics relies on the mixing of an analyte and ligand in the mobile phase immediately prior to entering the sensing region where one of the interactants (either the analyte or ligand) can also bind competitively to ligand immobilized on one of the sensing surfaces 25a, 25b or 25c. The dynamic competition between analyte binding to solution phase ligand and to solid phase ligand is encoded in the shape of the resulting solid phase binding response curve. A solution phase competitive kinetic model, or affinity model, may then be fitted to this solid phase data and both the solution phase and solid phase kinetic constants determined.

It may be desirable to delay time between sample mixing and arrival in the flow cell in order to generate high quality data. A steady state version of the competitive kinetic model is available that does not rely on a short lag time between mixing and arrival at the flow cell but it is desirable to have both options available. This type of injection can be performed as described above with respect to FIG. 1, but line 21 entering the junction 28 contains a liquid carrying a second analyte or free solution phase ligand. A stepped injection can be applied in this application. This type of injection is also very useful where reagents are highly sensitive to hydrolysis or degradation. For example, the activation of a carboxylic acid-coated surface is often performed using aqueous solutions of 0.4 M N-Ethyl-N′-(3-dimethylaminopropyl) carbodimide and 0.1 M N-hydroxysuccinimide that are mixed in a 1:1 ratio immediately before injecting over the sensing surface. The activity of these reagents can drop as much as 50% if they are pre-mixed 10 minutes before the injection. To avoid any degradation both reagents can be loaded separately and merged in a 1:1 flow ratio using the in-situ dilution method. Reagents that are prone to hydrolysis, or are unstable when diluted, may also be handled more appropriately by mixing immediately before delivery to the flow cell.

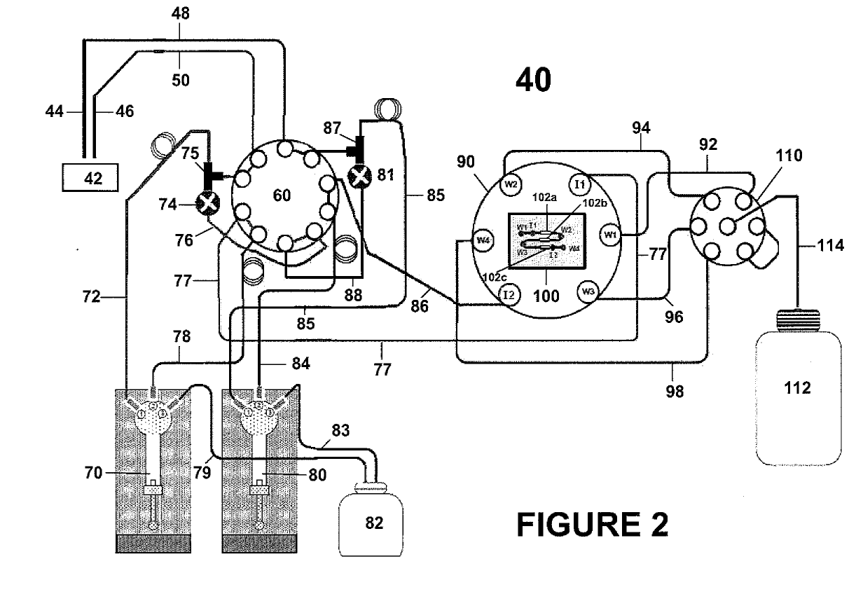

The inventive method can be performed using a wide variety of flow injection fluidic systems that include pumps, flow channels and valves in various arrangements. However, the fluidic configuration depicted in FIGS. 2 & 3 provides a preferred embodiment of a fluidic system. In this embodiment, multiple sensing surfaces can be fluidicly addressed with sample while using a minimum of active flow control devices.

As depicted in FIGS. 2 & 3, the fluidic system consists of the following components: autosampler 42; sample probes 44, 46; sample loading lines 48, 50; sample holding lines 72, 85; T-junctions 75, 87 with attached check valves 74, 81, respectively; router valve 60; jumper lines 76, 88; syringe pumps 70, 80 each with a 4-way distribution valve; buffer supply 82; inject lines (77, 86); microfluidic adapter 90 with sample input ports I1, I2 and waste ports W1, W2, W3, W4; sensing region 100 housing sensing surfaces; waste stream selector 110 and associated valves; and main waste line 114. Microfluidic adapter 90 enables free space tubing to make fluid contact with the serpentine microfluidic sensing region 100 with minimal dead volume, preferably less than 5 μl. Sensing region 100 houses three sensing surfaces 102a, 102b, 102c in series. Waste selector valve 110 allows a single waste to be open at any time thereby controlling the flow direction within sensing region 100 while the pumps 70, 80 allow two input streams (through input ports I1, I2) to enter sensing region 100 at any time or to combine in advance of entering the sensing region 100.

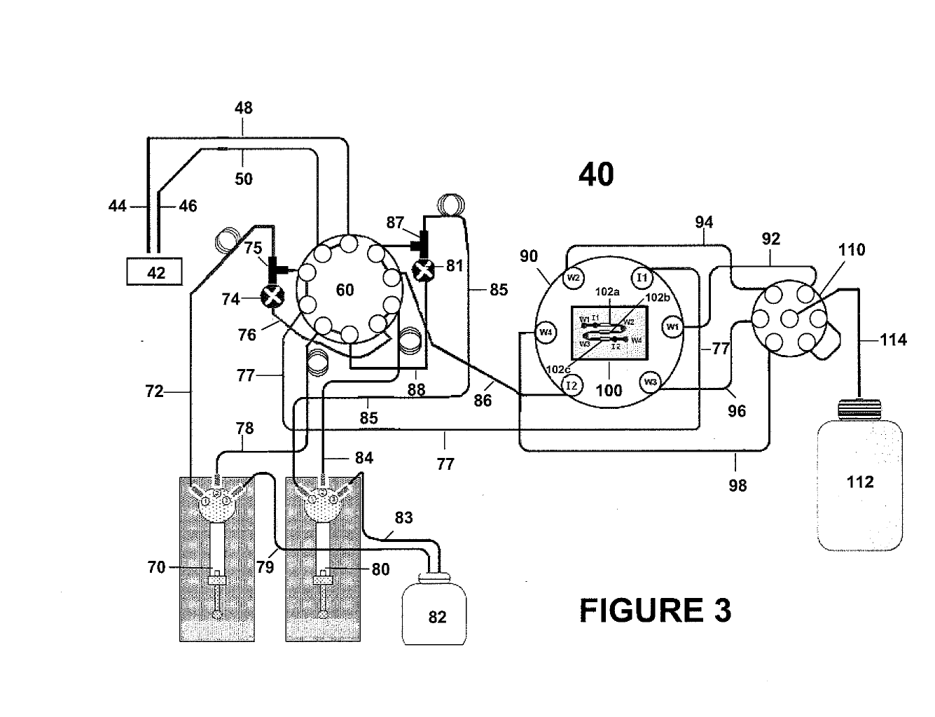

The in situ dilution method is initiated when pump 70 aspirates sample along flow path line 50 to line 72 while the router valve 60 is in the sample load position as shown in FIG. 2. The entire sample volume is pulled into the holding line 72. Concurrently, pump 80 supplies continuous flow buffer (i.e. analyte free liquid) into the serpentine sensing region 100 via flow path defined by line 84 to line 86 to input port I2 to waste port W1. The injection commences with 100% analyte free buffer such that the flow path defined by check valve 74 to T-junction 75 to line 77 must be washed out to remove any residual sample that remains in this path after sample loading. To begin preparing for the injection of sample (containing analyte), the router valve 60 is switched to the inject position as shown in FIG. 3. A volume of buffer is dispensed along the flow path defined by pump 80 to line 84 to line 76 to check valve 74 to line 77 to input port I1 to waste port W1 while buffer is supplied into the opposite side of the serpentine sensing region along the flow path defined by pump 70 to line 78 to line 88 to check valve 81 to line 86 to input port I2 to waste port W1. It is important to note that both streams exit at waste port W1 thereby preventing the washings from contacting the three sensing regions disposed in series between I1 and I2.

The analyte injection then commences by continuing to dispense buffer along the flow path defined by pump 80 to line 84 to line 76 to check valve 74 to line 77 to input port I1 to waste port W1 while switching off the flow stream defined by flow path pump 70 to line 78 to line 88 to check valve 81 to line 86 to input port I2 to waste port W1 and switching the waste stream selector 110 to waste port W4. This causes the buffer stream to pass over all three sensing surfaces 102a, 102b, 102c within sensing region 100. The first analyte concentration enters the sensing region 100 by flowing at a very low flow rate through the flow path defined by pump 70 to line 72 to line 77 to input port I1 to waste port W4 while buffer flows at a higher rate through the flow path defined by pump 80 to line 84 to line 76 to check valve 74 to line 77 to input port I1 to waste port W4. Both streams merge at the junction of line 72 and line 76, and become well mixed before entering input port I1 because of chaotic advection and turbulent flow that is induced by the tortuous flow path from the router valve 60 through the microfluidic adapter 90 into the sensing region 100.

It one aspect of this embodiment, the volumetric flow rate can be held constant and this can be accomplished by matching any incremental flow rate increase from pump 70 with an equivalent flow rate decrease in pump 80. Each time the flow rates are adjusted a change in analyte concentration occurs. The number of steps and the stepping frequency are adjusted to give the desired analyte injection profile with respect to injection time. The injection is terminated by switching to waste port W1 and resuming buffer flow along flow path defined by pump 70 to line 78 to line 88 to check valve 81 to line 86 to input port I2 to waste port W1. The combined stream that previously flowed through the sensing region 100 will now exit at waste port W1 causing a fast liquid exchange from analyte to buffer over all sensing regions.

The number of flow rate adjustments that can be made during a sample injection is largely dependent on the pump technology. Pumps which have the capacity to allow for smaller incremental increases or decreases in flow rate, such as high resolution syringe pumps with a wide stepping speed range, will provide a greater number of flow rate adjustments. Again, this range of flow rate adjustments is dependent on pump technology; however, the number of flow rate adjustments on pumps currently available is between 1 and 1,000. Pumps with greater flow rate adjustment capacity provide the benefit of being able to test a greater range of concentrations per sample injection. For example, the concentration of sample in a stream could increase stepwise over 1 to 6 orders of magnitude with a single injection based on a corresponding pump capacity to allow for such a change.

While it is preferable to hold the volumetric flow rate through the flow cell constant during the execution of the injection to create a full dose response it is nevertheless possible to complete a kinetic analysis where the flow rate is variable. For example, it is possible to widen the dose response range by allowing the buffer supply pump to pump at a very high flow rate for the first step so that the flow rate differential between the analyte and buffer pumps is maximized. If the minimum flow rate for a pump is 2 uL/min and the analyte pump is ran at this rate then one could run the buffer pump at 498 ul/min giving a dilution factor to 1/250. Thus a dose response range of 250-fold is enabled. However at these flow rates the final few steps consume a prohibitively large volume of sample and so it is desirable to ramp the volumetric flow rate downwards with respect to increasing steps.

To analyze a data set for analyte binding rescored in this fashion, it is necessary to ensure that the double referencing method accurately accounts for any temperature and pressure changes. This can be accomplished by conventional subtraction of a response curve from a reference surface and a response curve from a blank injection. The maintenance of isothermal conditions is not essential, but attention to reducing temperature gradients within the analysis chamber and allowing injected liquids to reach thermal equilibrium before reaching the flow cell is important to improving data quality with flow rate variations. When fitting a simple 1:1 pseudo-first-order interaction model, it is not necessary to account for the variable flow rate and it is assumed that the analyte binding interaction is independent of mass transport to the surface. In this case the data can be fitted as described for a fixed flow rate gradient injection. However, if mass transport is non-negligible then it is important to use a two-compartment model. However the two compartment model would need to be altered to allow a different Kₘ term for each step in the stepped injection. The Kₘ is the mass transport constant and is related to the analyte mass transport coefficient which is a function of flow rate. This change will allow the model to fit the data set accurately with minimal increase in fitting error with respect to fitting a conventional fixed flow rate stepped injection.

The flow control procedure for performing the in situ dilution step injection using the described fluidic system has been with reference to step analyte injections from holding line 72. However, the fluidic system is symmetrical and allows an equivalent step analyte injection to be performed from holding line 85 by repeating the exact procedure for holding line 72 but substitute all flow paths and waste selections with the symmetrical counterpart flow paths and waste positions.

There are many modifications to the above fluidics design and control procedure that could be implemented as an alternative to the preferred embodiment while retaining the benefit provided by the inventive method. For example, it is possible to substitute the syringe pumps with non-linear flow pumps of US2007/0183928 A1 or U.S. Pat. No. 5,942,093, both of which are incorporated herein by reference, to produce controlled non-linear analyte gradient profiles where the incremental step increases in analyte concentration are very small and occur very quickly enabling a better approximation of a continuous smoothly changing analyte gradient to be generated.

In addition, increasing the number of sensing surfaces 102 can be accomplished by also scaling the associated flow lines and control valves for equivalent operation. In the design provided in FIGS. 2 and 3 sensing surfaces 102a and 102c are independently addressable while the middle sensing surface 102b can only be fluidicly addresses by first passing over another sensing surface, either 102a or 102c. Addressing multiple sensing surfaces 102 at once, or allowing independently addressable sensing surface flow paths are options that are readily incorporated. The addition of an autosampler with 4,6,16 or 32 sample probes and the capability of concurrently injecting samples loaded from such a multichannel autosampler can also be accomplished by simply scaling the fluidic system and its components appropriately without any modification to the principles of the design. It is also apparent that a biosensing system possessing two, or more sensing surfaces in parallel, or in series, might each be addressed by fixed concentration injections and step analyte injections.

An alternative approach where two or more sets of two or more, sensing channels may be addressed individually by a step analyte injection but the individual sensing regions within each set are addressable by conventional fixed concentration injections. It is appreciated that other combinations of step analyte injections, fixed analyte injections and their respective allowable flow paths over two or more sensing regions are capable of being implemented into the core concept of the current inventive method.

It is possible to accomplish the capabilities of the design shown in FIGS. 1, 2 and 3 using alternative designs. In an alternative embodiment the functionality of the fluidics device described in FIGS. 1, 2 and 3 can be implemented in an integrated microfluidic circuit with embedded flow controls, or optionally, an integrated microfluidic circuit that is in fluid contact with remote peripheral flow control devices. U.S. Pat. No. 6,004,515, U.S. Pat. No. 6,149,870, U.S. Pat. No. 6,475,441B1 and WO/1998/056505, all of which are incorporated herein by reference, describe an electro osmotic microfluidic device for combining two, or more, liquids in a microfluidic system for the purpose of dilution. This approach could be applied as a suitable means of moving and intersecting two, or more, flow streams for the purpose of practicing the in situ dilution step injection method.

It is also possible to use two non-linear pumping mechanisms that produce two streams that can be made to merge and mix in order to prepare analyte gradients. In fact US 2007/0183928 A1, which is incorporated herein by reference, describes a pumping mechanism that can provide variable pressure to a flow stream thereby changing the flow rate. A flow meter measures the flow rate and the electronic controller then adjusts the pressure of the pressure transducer to bring the flow rate to the set flow rate. This process is dynamic and very fast allowing rapid changes (<1 sec) in flow rate to be performed.

This method also has the potential to reduce pulsation compared to syringe drive pumps. U.S. Pat. No. 5,942,093, which is incorporated herein by reference, describes a similar gradient making system where the non-linear flow is generated by applying the principle of electro osmotic flow where the voltage potential applied to each stream is controlled by applying a time dependent voltage profile to cause an equivalent flow rate profile in each controlled stream. Although this is a suitable means of generating flow streams with variable and controllable flow rates as a function of time it is dependent on the carrier liquid composition and is less desirable when compared to pressure driven flow.

The embodiments of the current invention relates to an in situ-dilution method that enables step injections of a sample to be performed where the concentration of sample steps up, or down, according to a predefined controlled profile without reliance on mixing alternating segments of sample and diluent through dispersion along the flow channels. Additionally, the invention provides a method to replace the multiple sample injections required by all other methods with a single injection whose sample concentration is modulated en route to the flow cell. This eliminates the overhead associated with multiple loading, injecting and clean up cycles that are necessary for prior art step-up analyte binding curves while also making it possible to completely eliminate unnecessary intermission periods. The dissociation of analyte can be accurately estimated from the single dissociation phase curve recorded after the step injection is complete thus a higher throughput can be achieved. The current invention eliminates the need for preparing dilutions sets thereby reducing assay complexity. This improves the holding capacity of the sample rack while reducing assay complexity substantially. The sample throughput is expected to decrease substantially compared to prior art methods.

Additionally, this invention reduces dependence on regeneration but more significantly eliminates the need for a dilution set altogether thereby reducing the overhead associated with multiple injections and the built-in “house keeping” routines (these are necessary to prevent cross-contamination and assure reproducible performance of the flow injection system) that accompany these injections. Sample dilution positions/vials are not required by the current invention allowing a 6-10 fold increase in analyte holding capacity in the sample racks. This may be expected to result in a 3-5 fold decrease in assay run time compared to the step-dilution series method and a 9-15 fold decrease compared to conventional methodology.

Additionally, the prior art methods fail to provide an increase in throughput as screening a large number of analyte compounds from libraries has become standard in drug discovery. The current invention addresses this problem by providing a method to decrease both the complexity and running time by incorporating an in-situ dilution making capability. The practice of preparing/loading/injection of each analyte dilution separately is eliminated in the current invention by loading the entire undiluted analyte sample into a holding line within the fluid system and then injecting from that holding line such that it merges and is diluted by a second stream that intersects the injection channel immediately before the flow cells (i.e. detector or sensing area(s)). By combining the analyte liquid flow with an analyte free flow, where the relative flow of each stream can be controlled accurately, it is possible to control the analyte concentration that enters the flow cell.

In contrast with other prior art methods, the current invention does not rely on pulsing between alternating segments of analyte containing fluid and the analyte free fluid. The pulse method requires significant dispersive mixing along the flow direction in order to bring about adequate mixing before reaching the flow cell. However, accurate estimation of the analyte concentration at any point after significant dispersion takes place is a complex function of molecular weight, viscosity, ionic strength, isoelectric point, channel geometry, flow rate and cannot be described by Taylor dispersion theory in the biosensor fluidic designs described. Therefore, the analyte gradient concentration is not precisely defined and can only be approximated by making assumptions and under special case conditions with no general solution. Eliminating any dependence on “Taylor-like” dispersion in the flow channels avoids these difficult problems yet all the gradient injection methods described in the previous prior art rely heavily on this means of mixing. Some level of dispersion will occur in most flow injection systems. However, for the purpose of correct operation of the step analyte injections, this dispersion should not be allowed to occur to such a degree as produce analyte injection profiles that are a significant function of molecular weight or other physical parameters of the analyte or fluidics.

The current invention can be used to form an analyte concentration gradient without relying on mixing by dispersion. In one embodiment of this invention, the analyte containing liquid (i.e. sample) intersects with the analyte free liquid (i.e. buffer) immediately before reaching the flow cell. The high resolution pumps that drive each flow stream are made to step incrementally during the analyte injection such that the concentration of analyte at the beginning of the injection is zero and increases as the injection proceeds. Instead of pulsing each line and then relying on dispersion to mix the alternating sample-buffer segments, as in prior art, the two laminar flows are instead merged, and mixed, in a very low dead volume (<5 μL) before reaching the flow cell. Under these conditions the concentration of the analyte in the flow cell can be calculated from the flow rate ratio at the merging junction and the neat analyte concentration.

The current inventive in-situ dilution method has widespread utility in many applications. For example, regeneration scouting is an iterative approach to finding liquid compositions that are capable of disrupting a given affinity complex while retaining the ligands binding activity. Typically this is done by preparing a series of dilutions of liquids containing various acids, bases, chaotrophic agents, solvents, electrolytes (or combinations of these reagents) and then injecting each from low-to-high potency. The application of the inventive in situ-dilution method would simplify this by eliminating the need for making discrete sample dilutions and also reducing the lag time between the serial injections. More significantly, it is possible to place a low potency liquid in one stream and a high potency liquid in the second stream and then both streams can be merged to form a stepped sample injection where the potency is variable throughout the injection. In this way a single injection can be used to screen over a large potency range with improved resolution. Scouting applications also include determination of the correct pH to enable effective charge pre-concentration and covalent coupling of biomolecules to a sensing surface. For example, a series of 10 mM sodium acetate buffers is usually prepared at a pH range of 3.0-5.5 (in increments of 0.5 pH units) where each contains a fixed concentration of ligand (e.g. 10 μg/ml). The samples are then injected in series in order to determine the optimum pH. This procedure can be simplified by replacing the entire injection series with a single gradient injection where the samples at each end of the pH range are merged stepwise to form the gradient. The Henderson-Hasselbalch equation can then be applied to calculate the pH at all times during the gradient. This procedure provides greater pH resolution than a fixed concentration injection series and can be completed in a fraction of the time.

### EXAMPLES

### Example 1: Validation of the In Situ Dilution Method for Determining Association and Dissociation Constants

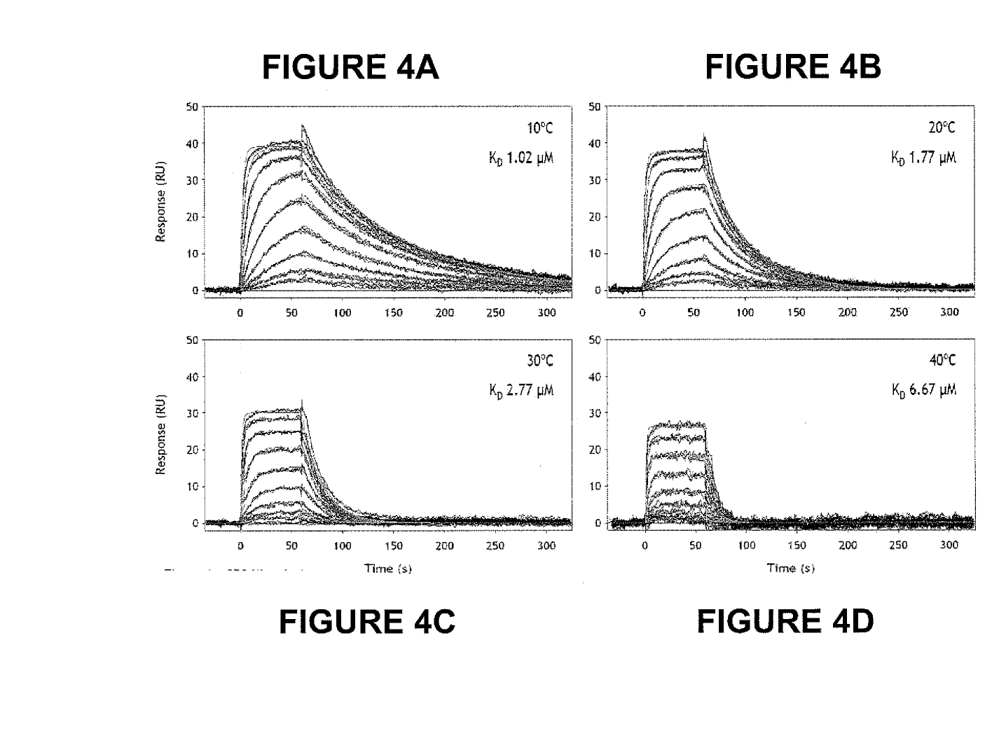

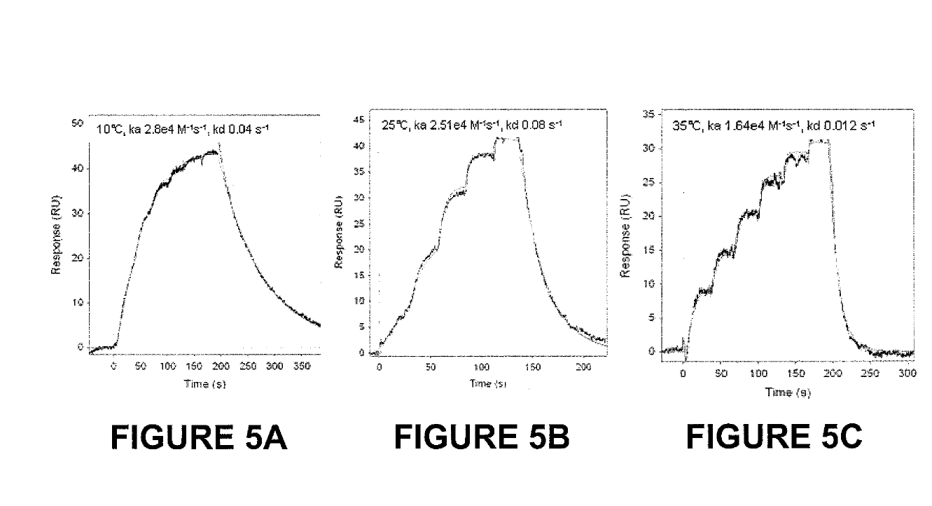

In Example 1, the kinetic values for the interaction between carboxybeneze sulfonamide and immobilized carbonic anhydrase were determined using a standard multiple injection method and the in situ dilution method described herein. In the classical approach sequential injection method (FIGS. 4A-4D) a series of individual concentrations were prepared as a serial doubling dilution set starting from a neat concentration of 40 mM giving a total of nine different concentrations. These concentrations were made up in the running buffer (i.e HEPES buffer saline, pH 7.4). 50 mL of each sample was injected at 50 mL/min and allowed dissociate for 300 seconds. Each concentration was repeated in duplicate and the entire analysis was repeated at four different analysis temperatures (10 C, 20 C, 30 C, 40 C—FIGS. 4A-D, respectively). FIG. 5 shows this analysis repeated using the in-situ dilution method. In this case a total of three interaction curves is recorded at three different temperatures representing a minimal data set that can be recorded at least an order faster than the conventional approach even normalized for the number replicates and the number of temperatures used. A single neat sample (40 mM) was injected using the in-situ-dilution injection and a serial doubling dilution of analyte was performed on-the-fly yielding a dose response curve. Dissociation was monitored for 200 seconds. This was repeated at three different temperatures. A blank injection was also included at each temperature for double referencing.

In both data sets the samples were injected over a protein coated and a non-coated surface to enable double referencing of the binding response curves. The resulting referenced binding response curves were then fitted with a kinetic 1:1 binding interaction model and the fitted curves are shown in red. The kinetic values were then analyzed thermodynamically to determined thermodynamic constants for the interaction and these are shown in Tables 1 and 2 below.

TABLE 1
Thermodynamic constants produced using
the traditional injection method.

| | ΔG^{‡} | ΔH ^{‡} | ΔS ^{‡} |
|---|---|---|---|
| Interaction | (kcal/mol) | (kcal/mol) | (cal/mol K) |
| Association | 11.60 | 3.96 | −26.1 |
| Dissociation | 19.32 | 14.63 | −16.0 |

TABLE 2
Thermodynamic constants produced using
the in situ dilution method.

| | ΔG^{‡} | ΔH ^{‡} | ΔS ^{‡} |
|---|---|---|---|
| Interaction | (kcal/mol) | (kcal/mol) | (cal/mol K) |
| Association | 11.1 | 4.1 | −24 |
| Dissociation | 18.8 | 13.1 | −20 |

As can be appreciated by studying Tables 1 and 2, the thermodynamic parameter values produced by the in situ dilution method are in excellent agreement with results from the conventional fixed concentration data set. Furthermore, the analysis can be completed in a fraction of the time by using the in situ dilution method. The in situ dilution method tested all concentrations in about 200 to 350 seconds, whereas the traditional injection method took approximately 2700 seconds to test all analyte concentrations.

### Example 2: Measuring Stepped (50 Steps) Increase in Analyte Concentrations Using the In-Situ Dilution Method

The sample in this case is water containing dimethylsulfoxide (DMSO) while the buffer pump was primed in water. The SPR based biosensor responds to the increase in refractive index as the concentration of DMSO increases. The sample pump flow rate and the buffer pump flow rate were incrementally adjusted inversely to each other while the volumetric flow rate through the flow cell was set as if to be held constant. The ideal step injection curve (linear) assuming no-air compliance and the recorded step injection bulk refractive index curve (non-linear) where a 20 μL air segment was placed within the sample injection line to cause delayed sample stream flow.

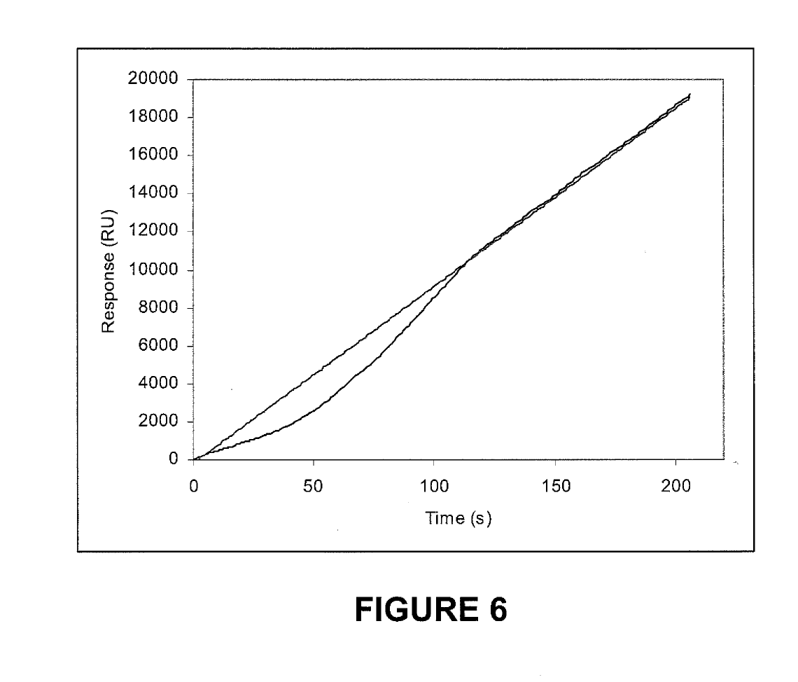

The injections described herein are preferably performed using liquid pumps with linear stepping action where the flow rate increases linearly with a linear increase in drive rate. This is the behavior expected from precision syringe pumps. However, compliance can be introduced such that the steady-state pressure head is not attained quickly. In this case the compliance will cause a non-linear flow to develop despite linear actuation of the pumps. This principle can be exploited to generate exponential segments in the analyte gradient that can increase the diversity of possible gradient profiles. With reference to FIG. 1, the insertion of an in-line air segment between the pump 14 and holding line 20 could be used to generate an exponentially changing sample stream flow rate due to the compressibility of the air segment. The linear buffer flow stream will then merge with the exponential sample flow stream resulting in an exponential analyte gradient injection at the flow cell. It will be difficult to hold the volumetric flow through the flow cell constant during such an injection but this is not essential in kinetic analysis of the data. A 50 step linearly increasing bulk refractive index injection was performed with air incorporated resulting in the response curve shown in FIG. 6. The lower region of the gradient injection deviates from linearity due to compliance of the air during the injection. This type of stepped gradient injection is useful where higher analyte resolution is required at lower concentrations. Alternative means of introducing compliance such as incorporation of expandable tubing might be used to similar effect.

The above description illustrates how compliance can be used to produce non-linear flow to great advantage. However, if linear flow rates are required, it is important to minimize compliance in the system. For example, the use of large air bubbles to prevent dispersion when loading, and injecting, samples must be eliminated. Reducing the total air volume to <3 μL may be an adequate compromise resulting in limited dispersion and relatively linear sample flow rates. If linearity must be maintained to a very high degree then it is necessary to eliminate the air completely and loading more sample than is required for the injection can provide a solution. Only the non-dispersed segment of the loaded sample volume is used in the injection and the excess, potentially dispersed sample remains in the holding line after the injection is complete. To this end, a larger sample volume is aspirated such that the sample region that suffers from mixing by dispersion can be ignored while the unmixed region can be used during the step-analyte injection. This method is effective but does consume as much as 2-fold more sample.

An alternative method is to substitute a non-miscible, non-compressible liquid in place of the air bubbles. This would assure linear flow rates while reducing dispersion considerably. There would be added overhead in terms of purging this non-miscible liquid from the fluidic system after an injection but could be implemented without difficulty. A desirable liquid must be water immiscible, be non-toxic, be resistant to dispersion when exposed to low levels of surfactants, should have low affinity for the tubing walls and should move as a discrete segment without break-up during flow when inserted into an aqueous flow stream.

### Example 3: The Effect of Dispersion on Kinetic Analysis

As stated earlier, dispersion can give rise to unpredictable analyte concentration profiles and any error in the analyte concentration will give rise to a corresponding error in the kinetic analysis. It is therefore desirable to use flow control and mixing techniques that do not rely on dispersion. In more detail, if an analyte is injected into a tube flowing under laminar flow conditions then it will tend to mix with the analyte free liquid already present in the tube. Assuming pressure driven flow, as the analyte segment travels along the tube it will become diluted due to the combined effect of molecular diffusion “smeared out” by the parabolic convective flow profile within the tube. This is used to great advantage to produce sigmoidal shaped analyte gradient injections (such as in WO 2004/109295 A1 and W02009/025680), but it is difficult to model the analyte concentration accurately when the dispersion volumes are low to intermediate as is the case in current biosensor designs.

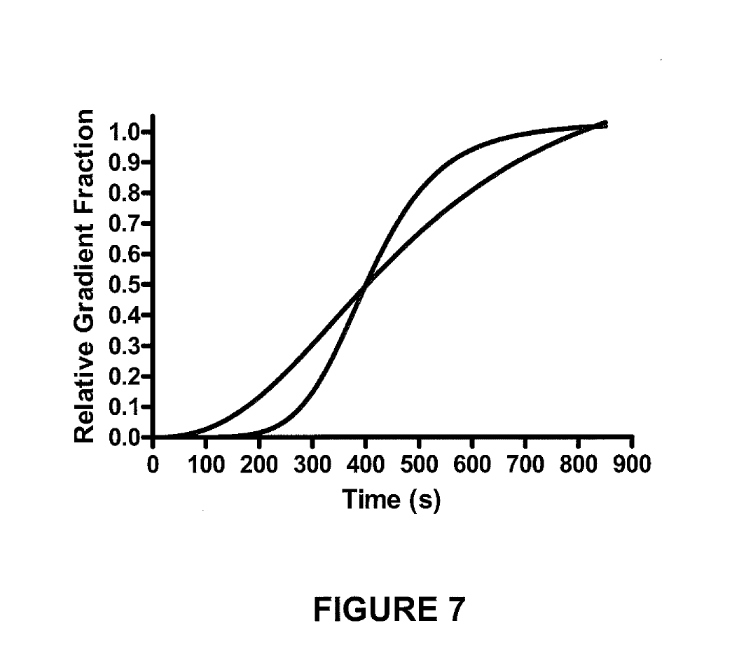

FIG. 7 depicts dispersion gradient profiles of two different analyte molecules as detected by refractive index sensitive surface plasmon resonance sensing. The steeper curve is for the protein macroglobulin (molecular weight 725 kDa) and the more gradual curve was recorded for bovine serum albumin (66 kDa). The proteins were prepared at a concentration of approximately 2 mg/ml in order to provide a well resolved bulk refractive index response. The bulk refractive index response was normalized to 1.0 at the maximum bulk refractive index response to aid comparison.

As demonstrated in FIG. 7, different analyte gradient profiles when injected using Taylor dispersion as a mixing method. There are no biomolecular binding interactions taking place in this data set and the response is generated by the change in refractive index that occurs when the analyte moves into the evanescent field (extends about 200-300 nm from the surface) at the sensing surface within the flow cell. In this example the flow rates, temperature and buffer composition were all held constant so that that variation with respect to molecular weight could be determined. The large variation between the gradient profiles observed in FIG. 7 is therefore due to the molecular weight dependency of Taylor dispersion. Such molecular weight dependency can be modeled by Taylor dispersion theory if a long high volume dispersion channel is used but this approach is contrary to the goal of preparing an analyte gradient profile that is invariant with the identity of the analyte and this does simplify data analysis.

Therefore the advantages of the gradient injection methods proposed in prior art cannot be fully achieved for all analytes. However, the embodiments of the current invention provides in-situ dilution capability which can be used to form an analyte concentration gradient without relying on mixing by dispersion and therefore produces a universal gradient profile that is independent of the analyte's biophysical characteristics. In one embodiment of the current invention, the analyte containing liquid (i.e. sample) intersects with the analyte free liquid (i.e. buffer) immediately before the flow cell. The high resolution pumps that drive each flow stream are made to step incrementally during the analyte injection such that the concentration of analyte at the beginning of the injection is zero and increases as the injection proceeds. Instead of pulsing each line and then relying on dispersion to mix the alternating sample-buffer segments, as in prior art, the two laminar flows are instead merged, and mixed, in a very low dead volume (<5 μL) before reaching the flow cell. Under these conditions the concentration of the analyte in the flow cell can be calculated from the flow rate ratio at the merging junction and the neat analyte concentration. For example, if a linear analyte gradient is desired then the buffer pump begins at a maximum flow rate while the sample pump remains off. The sample pump then begins delivering sample at a very low flow rate while the buffer pump flow rate is reduced by an equivalent rate. The sample pump will continue to step up in flow rate linearly while the buffer pump will step down synchronously by the same increments. Thus the sample flow and buffer flow are inversely related to each other and allows a constant volumetric flow to be delivered to the flow cell while the analyte concentration increases step-wise.

### Example 4: Measured Stepped (50 Steps) Increase In Sample Concentration Using the In-Situ Dilution Mechanism

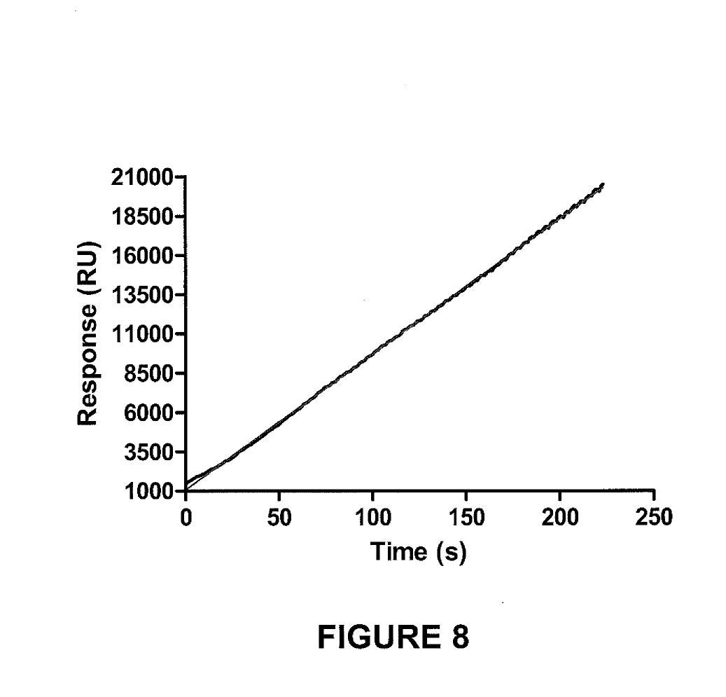

FIG. 8 shows a measured stepped sample injection performed using the inventive in-situ dilution method where there were 50 stepped increases in sample concentration over the progress of the injection. The smooth line is a fitted linear model giving an R2 of 0.9996. The sample is water containing DMSO while the buffer pump was primed in water. The SPR based biosensor responds to the increase in refractive index as the concentration of DMSO increases. The sample pump flow rate and the buffer pump flow rate were incrementally adjusted inversely to each other while the volumetric flow rate through the flow cell was held constant.

A linear pump stepping profile resulted in the linear stepped gradient injection profile. The periodic undulations in the recorded response curve are due to the linear step increases in sample concentration. It is important to point out that the data is well described by a smooth linear approximation. Therefore when the number of incremental steps is high it is possible to represent the gradient profile as a simple smoothly changing line as shown in FIG. 8. Non-linear gradient profiles may be similarly approximated. The number of steps may be increased such that the incremental change in concentration with each step is very small relative to the total change over the progress of the analyte injection thereby allowing the stepped pseudo-fixed concentration profile to be considered as a continuous analyte gradient injection. This approximation will be more appropriate for a large number of steps.

### Example 5: Stepped Bulk Refractive Index Injection (Blue Curve) Performed Using the In-Situ Dilution Method

It is also possible to avoid approximations by using a bulk refractive index curve (as in FIG. 8) to represent the gradient profile for the purpose of biomolecular interaction model fitting as described in WO/2009/02568. Importantly, the level of sample dispersion that is possible in a very low dead volume (as is used in this in-situ merging technique) is not sufficient to produce any significant Taylor dispersion. Therefore, the assumption that all analytes will behave similarly is reasonable and is in contrast to the prior art. Thus a stepped bulk index response can be assumed to be a reliable approximation of what can be expected from a stepped analyte injection of any analyte sample assuming the injection parameters are held constant. By using the bulk index method to calibrate the analyte concentration in the stepped analyte injection we can account for any subtle changes in the gradient profile that result from mixing in the dead volume, or changes due to pressure, flow rate or compliance within the fluidic system.

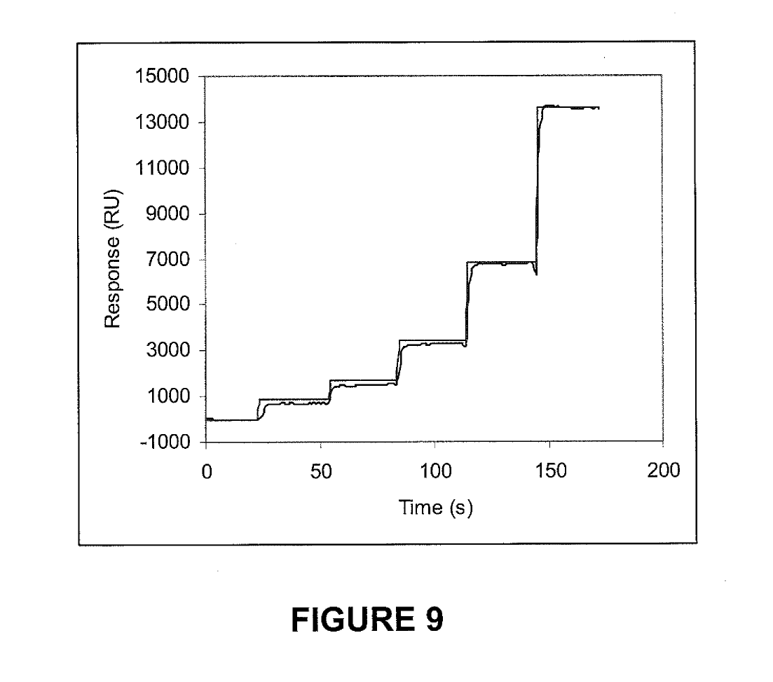

This method can be used with any stepped analyte injection including those with a low, or a high, number of steps during the injection. An advantage of the inventive method is that the binding interaction model can be reliably fit even during transition regions where the analyte concentration steps to a new steady state concentration. For example, FIG. 9 shows a stepped bulk refractive index injection performed using the in-situ dilution method according to a serial doubling dilution ratio. In FIG. 9, the biosensor is primed in water and then water containing DMSO was injected so that a series of five doubling dilutions of analyte were injected sequentially over the surface. A 3 μL air segment was introduced to cause compliance in the sample stream and results in a non-linear sample stream flow rate at the stream merging junction that is evident at lower dilutions.

The ideal stepped bulk refractive index response profile (smooth stair profile) is similar to the recorded analyte response profile but there is significant error (intentionally introduced) at the lower dilutions. This difference would not translate into any error, when fitting a kinetic or affinity model, if this stepped bulk index injection is used to calibrate the analyte concentration for a stepped analyte injection performed using the same injection parameters. This principle is outlined in detail in patent application WO/2009/02568, which is incorporated herein by reference.

However, the second method that calculates the concentration from the neat concentration and the flow rate ratio during stream merging would cause an avoidable error in the fitted model. This is especially true in regions where the analyte concentration transitions to the next in the series. There is a short lag time (rise) time where the concentration of analyte is not constant during the start of each analyte concentration step. Some methods used in the prior art require that the concentration of the individual analyte segments is known in advance of performing a stepped analyte titration. In particular, there is no provision for the transition segments where the analyte concentration is in transition from one steady-state concentration to the next. In contrast the bulk refractive index method of patent application WO/2009/02568 allows the concentration of the analyte to be known at all times during a stepped analyte injection series. Therefore, the inventive in-situ dilution method does not need to precisely follow an exact dilution sequence assuming that the bulk refractive index calibration method is applied.

For example, one prior art method relies on manual, or autosampler-based, preparation of a dilution set from a neat sample of known concentration. The concentration of each dilution is then inferred from the type of dilution made. For example, if a serial doubling dilution set is prepared then the concentration will halve with each subsequent dilution. These distinct volumes of liquid are loaded from the sample rack and injected through the flow cell(s) as separate volumes. In practice there will be error associated with the liquid transfer steps, and liquid mixing steps, associated with preparing such a dilution series and these errors are not accounted for during model fitting. In fact, even if the concentration of each individual analyte dilution was independently determined in advance there would still exist a measurement error associated with each of these measurements in addition to a large analyte concentration error during the start of each injection in the series.

In contrast, the analyte concentration error will not propagate in the current in-situ dilution method because only the neat sample is loaded and injected. It is merging with a second liquid stream that brings about the dilution, and hence, the number of successive fluid manipulations is greatly reduced thereby reducing the associated error in analyte concentration. In addition to this improvement, a method that can compensate for any remaining error in analyte concentration such as the bulk index method of patent application WO/2009/02568 is a preferable means of estimating the analyte concentration when fitting binding interaction models.

Making the assumption that the analyte concentrations that flow through the flow cell are in fact known very accurately does not hold at all times during a stepped analyte injection series. This is of concern where the entire curve is to be fitted with a binding interaction model. A more serious error, that is usually ignored, is the assumption that the change in analyte concentration during transition from one analyte injection to the next in the series occurs infinitely fast. In practice this is not true and thus limits the kinetic range of the biosensing system. The analyte concentration within the flow cell is never identical to the concentration of neat analyte, or of any discrete dilution volumes, that can be made because of the dispersion effects that occur during all fluid manipulations. Also the concentration cannot undergo instantaneous transitions from one concentration to the next because these transitions are a gradual progression. Hence the analyte concentration within the flow cell is best described as being continuously variable with optional steady-state regions where the analyte concentration is relatively stable and can be approximated as being fixed. Therefore in the interests of fitting a binding interaction model to the entire stepped analyte injection curve (i.e. no regions omitted) the bulk refractive index method is preferable.

The analyte gradient profile as a function of time can still be approximately calculated from the neat analyte concentration, which is know a priori (which is usually the case), and the flow ratio at the merger of the sample stream and buffer streams. This method is expected to be reasonable when fitting to pseudo steady-state regions and the error associated with applying this method is often acceptable. However the use of the bulk index method has the advantage of including a more accurate description of the physical system and can also compensate for any unexpected contributions such as compliance within the fluidics as illustrated in FIG. 9.

In contrast to the prior art methods that are reliant on large scale dispersion, low level analyte dispersion within a low dead volume will be consistent for different analyte entities. While it is desirable to hold the volumetric flow rate through the flow cell constant it is not essential to the success of the method. Furthermore, the gradient profile is not limited to the very simple linear gradient profile illustrated in FIGS. 7-8. In fact almost any gradient profile can be obtained by suitable adjustment of the stepping rates over the course of the analyte gradient injection.

After appropriate referencing of the data, the stepped analyte binding curve can be directly fitted (preferably globally) with a binding interaction model in order to estimate kinetic and/or affinity constants. However, models that can be used to determine other parameters associated with the biomolecules may also be applied including active concentration, molecular weight, diffusion coefficients etc. The dual sample merging injection can be used in conjunction with a competitive solution phase kinetic model as well as a competitive solution phase affinity model. The referencing of binding interaction curves is somewhat different for gradient injections when compared to fixed concentration injections. For fixed concentration injections double referencing requires subtracting the response from an injection of a blank sample (i.e. identical to analyte sample but analyte is not present) from sample injections containing analyte. The response from an uncoated sensing surface is then subtracted. The response due to mass changes at the surface is thereby isolated and is ready for model fitting. On occasion it may be necessary to include solvents such as DMSO in the sample buffer in order to maintain the solubility of dissolved analytes. It is therefore not trivial to match the refractive index of the running buffer with the refractive index of the sample. When high ligand loading densities are used an artifact known as the excluded volume effect can remain in the data after double referencing. This artifact is caused by the difference in the bulk refractive index response over surfaces coated with different levels of ligand.

For example if the reference surface is uncoated and the working surface is coated with a high concentration of ligand then it is likely that an excluded volume response will remain in the data after double referencing. This excluded volume response is due to the having less liquid within the evanescent field on the densely coated sensing surface relative to the uncoated reference surface. There are two approaches to overcoming this artifact. (1) Immobilize an equivalent amount of non-sense protein onto the reference channel. (2) Generate a calibration curve (excluded volume response v's analyte concentration) for this effect by running DMSO standards along with samples and then this excluded volume contribution to the binding response curve can be subtracted. The response to be subtracted is a constant for each fixed concentration. However, a step injection, where the analyte concentration is variable, requires that the excluded volume response is determined from the calibration curve for all time points thereby generating a correction curve. This can be considered a third reference curve for triple referencing of the binding interaction curve.

Thus, the present invention is well adapted to carry out the objects and attain the ends and advantages mentioned above as well as those inherent therein. While preferred embodiments of the invention have been described for the purpose of this disclosure, changes in the construction and arrangement of parts and the performance of steps can be made by those skilled in the art, which changes are encompassed within the spirit of this invention as defined by claims when they are appended.

What is claimed is:

1. A method for determining interaction kinetics of an analyte over a range of dilutions comprising the steps:
providing a single concentration of said analyte;
providing a buffer suitable for diluting said analyte;
initiating injection of said analyte into a first fluid flow path at a first analyte injection rate such that said analyte passes through said first fluid flow path;
initiating injection of said buffer into a second fluid flow path at a first buffer injection rate such that said buffer passes through said second fluid flow path;
said first and second flow paths merge to form a third flow path, said third flow path having a dead volume, said analyte and said buffer passing from said first and second flow paths into said dead volume wherein said analyte and buffer form a homogenous mixture of analyte and buffer;
passing said first homogenous mixture of analyte and buffer through a sensing region having at least one sensing surface, said sensing surface having a ligand attached thereto; and,
determining the interaction kinetics of said analyte in said a homogenous mixture of analyte and buffer with said ligand.

2. The method of claim 1, wherein the dead volume is equal to or less than 5 μl.

3. The method of claim 1, further comprising the step of calculating the concentration of the analyte in the resulting homogenous mixture of analyte and buffer based on the initial concentration of analyte injected into the first fluid flow path, the flow rate of the initial concentration of analyte through the first fluid flow path and the flow rate of buffer through the second fluid flow path.

4. The method of claim 1, wherein the mixing of analyte and buffer within said dead volume occurs under conditions which preclude formation of a dispersion.

5. A method for determining interaction kinetics of an analyte over a range of dilutions comprising the steps:
providing a single concentration of said analyte;
providing a buffer suitable for diluting said analyte;
initiating injection of said analyte into a first fluid flow path at a first analyte injection rate such that said analyte passes through said first fluid flow path;
initiating injection of said buffer into a second fluid flow path at a first buffer injection rate such that said buffer passes through said second fluid flow path;
said first and second flow paths merge to form a third flow path, said third flow path having a dead volume, said analyte and said buffer passing from said first and second flow paths into said dead volume wherein said analyte and buffer form a first homogenous mixture of analyte and buffer;
passing said first homogenous mixture of analyte and buffer through a sensing region having at least one sensing surface, said sensing surface having a ligand attached thereto;
continuing the injection of said analyte into said first fluid flow path at a second analyte injection rate such that said analyte passes through said first fluid flow path;
continuing the injection of said buffer into said second fluid flow path at said first buffer injection rate or at a second buffer injection rate such that said buffer passes through said second fluid flow path;
mixing said second analyte injection with said second buffer injection in said dead volume to provide a second homogenous mixture of analyte and buffer;
passing said second homogenous mixture of analyte and buffer through a sensing region having at least one sensing surface, said sensing surface having a ligand attached thereto; and,
determining the interaction kinetics of said analyte in said first and second homogenous mixtures of analyte and buffer with said ligand.

6. The method of claim 5, wherein the dead volume is equal to or less than 5 μl.

7. The method of claim 5, further comprising the step of calculating the concentration of the analyte in the resulting homogenous mixture of analyte and buffer based on the initial concentration of analyte injected into the first fluid flow path, the flow rate of the initial concentration of analyte through the first fluid flow path and the flow rate of buffer through the second fluid flow path.

8. The method of claim 5, wherein the mixing of analyte and buffer within said dead volume occurs under conditions which preclude formation of a dispersion.

9. The method of claim 5, wherein the analyte flow through said first fluid flow path under laminar flow conditions and said buffer flows through said second fluid flow path under laminar flow conditions.

10. A method for determining interaction kinetics of an analyte over a range of dilutions comprising the steps:
providing a single concentration of said analyte;
providing a buffer suitable for diluting said analyte;
initiating injection of said analyte into a first fluid flow path at a first analyte injection rate such that said analyte passes through said first fluid flow path;
initiating injection of said buffer into a second fluid flow path at a first buffer injection rate such that said buffer passes through said second fluid flow path;
said first and second flow paths merge to form a third flow path, said third flow path having a dead volume, said analyte and said buffer passing from said first and second flow paths into said dead volume wherein said analyte and buffer form a first homogenous mixture of analyte and buffer;
passing said first homogenous mixture of analyte and buffer through a sensing region having at least one sensing surface, said sensing surface having a ligand attached thereto;
continuing the injection of said analyte into said first fluid flow path at said first analyte injection rate or at a second analyte injection rate such that said analyte passes through said first fluid flow path;
continuing the injection of said buffer into said second fluid flow path at a second buffer injection rate or if said second injection of said analyte occurs at a second analyte injection rate injecting said buffer at said first buffer injection rate such that said buffer passes through said second fluid flow path;
mixing said second analyte injection with said second buffer injection in said dead volume to provide a second homogenous mixture of analyte and buffer;
passing said second homogenous mixture of analyte and buffer through a sensing region having at least one sensing surface, said sensing surface having a ligand attached thereto; and,
determining the interaction kinetics of said analyte in said first and second homogenous mixtures of analyte and buffer with said ligand.

11. The method of claims 10, wherein the dead volume is equal to or less than 5 μl.

12. The method of claim 10, further comprising the step of calculating the concentration of the analyte in the resulting homogenous mixture of analyte and buffer based on the initial concentration of analyte injected into the first fluid flow path, the flow rate of the initial concentration of analyte through the first fluid flow path and the flow rate of buffer through the second fluid flow path.

13. The method of claim 10, wherein the mixing of analyte and buffer within said dead volume occurs under conditions which preclude formation of a dispersion.

14. The method of claim 10, wherein if the analyte injection flow rate increases, the flow rate of buffer decreases an incremental amount proportionate to the increase of analyte injection flow rate.

15. The method of claim 10, wherein if the analyte injection flow rate decreases, the flow rate of buffer increases an incremental amount proportionate to the increase of analyte injection flow rate.

16. The method of claim 10, wherein the analyte flow through said first fluid flow path under laminar flow conditions and said buffer flows through said second fluid flow path under laminar flow conditions.

17. A method for determining interaction kinetics of an analyte over a range of dilutions comprising the steps:
providing a single concentration of said analyte;
providing a buffer suitable for diluting said analyte;
initiating injection of said analyte into a first fluid flow path at a first analyte injection rate such that said analyte passes through said first fluid flow path;
initiating injection of said buffer into a second fluid flow path at a first buffer injection rate such that said buffer passes through said second fluid flow path;
said first and second flow paths merge to form a third flow path, said third flow path having a dead volume, said analyte and said buffer passing from said first and second flow paths into said dead volume wherein said analyte and buffer form a first homogenous mixture of analyte and buffer;
passing said first homogenous mixture of analyte and buffer through a sensing region having at least one sensing surface, said sensing surface having a ligand attached thereto;
continuing the injection of said analyte into said first fluid flow path at an analyte injection rate that differs from the first analyte injection rate such that said analyte passes through said first fluid flow path;
continuing the injection of said buffer into said second fluid flow path at a buffer injection rate that differs from said first buffer injection rate such that said buffer passes through said second fluid flow path;
mixing said second analyte injection with said second buffer injection in said dead volume to provide a second homogenous mixture of analyte and buffer;
passing said second homogenous mixture of analyte and buffer through a sensing region having at least one sensing surface, said sensing surface having a ligand attached thereto; and,
determining the interaction kinetics of said analyte in said first and second homogenous mixtures of analyte and buffer with said ligand.

18. The method of claim 17, wherein the dead volume is equal to or less than 5 μl.

19. The method of claim 17, further comprising the step of calculating the concentration of the analyte in the resulting homogenous mixture of analyte and buffer based on the initial concentration of analyte injected into the first fluid flow path, the flow rate of the initial concentration of analyte through the first fluid flow path and the flow rate of buffer through the second fluid flow path.

20. The method of claim 17, wherein the mixing of analyte and buffer within said dead volume occurs under conditions which preclude formation of a dispersion.

21. The method of claim 17, wherein the analyte flow through said first fluid flow path under laminar flow conditions and said buffer flows through said second fluid flow path under laminar flow conditions.

22. A method for determining interaction kinetics of an analyte over a range of dilutions comprising the steps:
initiating injection of a first concentration of analyte into a first fluid flow path at a first analyte injection rate such that said analyte passes through said first fluid flow path;
initiating injection of a buffer into a second fluid flow path at a first buffer injection rate such that said buffer passes through said second fluid flow path;
said first and second flow paths merge to form a third flow path, said third flow path having a dead volume, mixing said analyte and said buffer within said dead volume to provide a first mixture of analyte and buffer having a constant concentration;
passing said first mixture of analyte and buffer through a sensing region having at least one sensing surface, said sensing surface having a ligand attached thereto;
continuing the injection of said analyte into said first fluid flow path at a second analyte injection rate such that said analyte passes through said first fluid flow path;
continuing the injection of said buffer into said second fluid flow path at a second buffer injection rate such that said buffer passes through said second fluid flow path;
mixing said second analyte injection with said second buffer injection in said dead volume to provide a second mixture of analyte and buffer having a constant concentration;
passing said second homogenous mixture of analyte and buffer through a sensing region having at least one sensing surface, said sensing surface having a ligand attached thereto; and,
determining the interaction kinetics of said analyte in said first and second homogenous mixtures of analyte and buffer with said ligand.

23. The method of claim 22, wherein the dead volume is equal to or less than 5 μl.

24. The method of claim 22, further comprising the step of calculating the concentration of the analyte in the resulting homogenous mixture of analyte and buffer based on the initial concentration of analyte injected into the first fluid flow path, the flow rate of the initial concentration of analyte through the first fluid flow path and the flow rate of buffer through the second fluid flow path.

25. The method of claims 22, wherein the mixing of analyte and buffer within said dead volume occurs under conditions which preclude formation of a dispersion.

26. The method of claims 22, wherein the analyte flow through said first fluid flow path under laminar flow conditions and said buffer flows through said second fluid flow path under laminar flow conditions.

27. The method of claims 22, further comprising the step of determining the interaction kinetics of said first homogenous mixture of analyte and buffer with said ligand prior to continuing the injection of said analyte or said buffer at a second flow injection flow rate.
